(19) United States
(12) Patent Application Publication
(10) Pub. No.: US 2013/0197080 A1
(43) Pub. Date: Aug. 1, 2013

(54) USE OF TERIFLUNOMIDE FOR TREATING MULTIPLE SCLEROSIS

(71) Applicant: SANOFI, Paris (FR)

(72) Inventor: Ross ROCKLIN, Bridgewater, NJ (US)

(73) Assignee: SANOFI, Paris (FR)

(21) Appl. No.: 13/755,825

(22) Filed: Jan. 31, 2013

Related U.S. Application Data
(63) Continuation of application No. PCT/US2011/046076, filed on Aug. 1, 2011.
(60) Provisional application No. 61/369,779, filed on Aug. 2, 2010.

(51) Int. Cl. A61K 31/277

(52) U.S. Cl. CPC A61K 31/277
USPC 514/521

(57) ABSTRACT

This invention relates to use of teriflunomide for treating Multiple Sclerosis (MS) and managing the risks associated with the use of teriflunomide.

This application is a Continuation of International Application No. PCT/US2011/046076, filed Aug. 1, 2011, which claims the benefit of Provisional Application No. 61/369,779, filed Aug. 2, 2010, Provisional Application No. 61/369,780, filed Aug. 2, 2010, Provisional Application No. 61/389,375, filed Oct. 4, 2010, and Provisional Application No. 61/424,717, filed Dec. 20, 2010, all of which are incorporated herein by reference in their entireties.

### FIELD OF THE INVENTION

This invention relates to use of teriflunomide for treating Multiple Sclerosis (MS) and managing the risks associated with the use of teriflunomide.

### BACKGROUND OF THE INVENTION

Teriflunomide is a novel oral disease-modifying therapy (DMT) in development for the treatment of relapsing-remitting multiple sclerosis (RMS). Teriflunomide blocks de novo pyrimidine synthesis, which inhibits the replication and function of activated (but not resting) lymphocytes.

The compound of (Z)-2-cyano-3-hydroxy-but-2-enoic acid-(4′-trifluoromethylphenyl)-amide (also known as teriflunomide, Formula I) is described in U.S. Pat. No. 5,679,709.

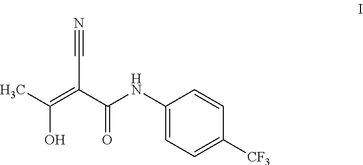 [SMILES: C/C(O)=C(\C#N)C(=O)Nc1ccc(C(F)(F)F)cc1]

The use of teriflunomide for treating multiple sclerosis is described in U.S. Pat. No. 6,794,410.

### SUMMARY OF THE INVENTION

This invention relates to a method for reducing annualized relapse rate (ARR) in a patient having relapsing form of multiple sclerosis in need thereof, comprising administering to the patient 14 mg of teriflunomide daily, wherein the method is clinically proven effective.

This invention also relates to a method for delaying disability progression in a patient having relapsing form of multiple sclerosis in need thereof, comprising administering to the patient 14 mg of teriflunomide daily, wherein the method is clinically proven effective.

### BRIEF DESCRIPTION OF THE DRAWINGS

FIG. 1 shows the patient disposition through the TEMSO study.

FIG. 2 shows the time to first relapse and patients remained free of relapse in the three patient groups in the TEMSO study.

FIG. 3 shows the 12-week-confirmed disability progression of the three patient groups in the TEMSO study.

FIG. 4 shows the mean number of T1-Gd-enhancing lesions (A) and proportion of patients free from T1-Gd-enhancing lesions (B) of the three patient groups in the TEMSO study.

FIG. 5 shows the number of unique active lesions per scan of the three patient groups in the TEMSO study.

FIG. 6 shows the change from baseline in burden of disease over time of the three patient groups in the TEMSO study.

FIG. 7 shows mean change from baseline in the volume of T1 hypointense lesions of the three patient groups in the TEMSO study.

### DETAILED DESCRIPTION OF THE INVENTION

As used above, and throughout the description of the invention, the following terms, unless otherwise indicated, shall be understood to have the following meanings:

“Clinically proven effective” means the efficacy of treatment has been proven by a Phase III clinical trial as statistically significant with an alpha level less than 0.05 or the clinical efficacy results are sufficient to meet approval standards of U.S. Food and Drug Administration.

“EDSS” means the Kurtzke Expanded Disability Status Scale.

“Patient” means humans.

One embodiment of the invention is related to a method for reducing annualized relapse rate by about 31.5% in a patient having relapsing form of multiple sclerosis in need thereof, comprising administering to the patient 14 mg of teriflunomide daily, wherein the method is clinically proven effective.

Another embodiment of the invention is related to a method for reducing the risk of disability progression in a patient having relapsing form of multiple sclerosis in need thereof, comprising administering to the patient 14 mg of teriflunomide daily, wherein the method is clinically proven effective.

Another embodiment of the invention is related to a method for reducing the risk of disability progression by about 29.8% progression in a patient having relapsing form of multiple sclerosis in need thereof, comprising administering to the patient 14 mg of teriflunomide daily, wherein the method is clinically proven effective.

Another embodiment of the invention is related to a method for reducing burden of disease in a patient having relapsing form of multiple sclerosis in need thereof, comprising administering to the patient 14 mg of teriflunomide daily, wherein the method is clinically proven effective.

Another embodiment of the invention is related to a method for reducing burden of disease by about 67.4% in a patient having relapsing form of multiple sclerosis in need thereof, comprising administering to the patient 14 mg of teriflunomide daily, wherein the method is clinically proven effective.

Another embodiment of the invention is related to a method for reducing the number of Gd-enhancing T1 lesions per scan in a patient having relapsing form of multiple sclerosis in need thereof, comprising administering to the patient 14 mg of teriflunomide daily, wherein the method is clinically proven effective.

Another embodiment of the invention is related to a method for reducing the number of Gd-enhancing T1 lesions per scan by about 80.4% in a patient having relapsing form of multiple sclerosis in need thereof, comprising administering to the patient 14 mg of teriflunomide daily, wherein the method is clinically proven effective.

Another embodiment of the invention is related to a method for improving proportion of patients free from Gd-enhancing T1 lesions in patients having relapsing-forms of multiple sclerosis in need thereof, comprising administering to the patient 14 mg of teriflunomide daily, wherein the method is clinically proven effective.

Another embodiment of the invention is related to a method for improving proportion of patients free from Gd-enhancing T1 lesions to about 64.1% in patients having relapsing-forms of multiple sclerosis in need thereof, comprising administering to the patient 14 mg of teriflunomide daily, wherein the method is clinically proven effective.

Another embodiment of the invention is related to a method for reducing the number of unique active lesions per scan in a patient having relapsing-forms of multiple sclerosis in need thereof, comprising administering to the patient 14 mg of teriflunomide daily, wherein the method is clinically proven effective.

Another embodiment of the invention is related to a method for reducing the number of unique active lesions per scan by about 69.4% in a patient having relapsing-forms of multiple sclerosis in need thereof, comprising administering to the patient 14 mg of teriflunomide daily, wherein the method is clinically proven effective.

Another embodiment of the invention is related to a method for reducing the T1 hypointense lesion volume in a patient having relapsing-forms of multiple sclerosis in need thereof, comprising administering to the patient 14 mg of teriflunomide daily, wherein the method is clinically proven effective.

Another embodiment of the invention is related to a method for reducing the T1 hypointense lesion volume by about 31.3% in a patient having relapsing-forms of multiple sclerosis in need thereof, comprising administering to the patient 14 mg of teriflunomide daily, wherein the method is clinically proven effective.

It is reported that elevation of liver enzymes, hypertension, infection and serious infection, bone marrow disorders, peripheral neuropathy and polyneuropathy have been observed during the TEMSO clinical trial. Thus, another aspect of the invention is related to managing the risks associated with the use of teriflunomide for treating multiple sclerosis.

Another embodiment of the invention is related to a method for managing the risk of elevation of liver enzyme to allow an effective and safe use of teriflunomide or a pharmaceutically acceptable salt thereof in the treatment of multiple sclerosis in a patient in need thereof comprising:
a) obtaining transaminase and bilirubin levels of the patient wherein the transaminase and bilirubin levels are measured within six month prior to the treatment; and
b) administering to the patient a pharmaceutically effective amount of teriflunomide or a pharmaceutically acceptable salt thereof.

To manage the risk associated with elevation of liver enzymes, the method can further comprise the following steps:
c) monitoring liver enzymes during the treatment; particularly to the patient who develop symptoms suggestive of hepatic dysfunction, such as unexplained nausea, vomiting, abdominal pain, fatigue, anorexia or jaundice and/or dark urine; and
d) if significant liver injury is suspected or if the elevation of liver enzymes greater than three times the upper limit of normal is confirmed, then considering discontinuing the treatment.

Another embodiment of the invention is related to a method of managing the risk of high blood pressure to allow an effective and safe use of teriflunomide or a pharmaceutically acceptable salt thereof in the treatment of multiple sclerosis in a patient in need thereof comprising:
a) administering to the patient a pharmaceutically effective amount of teriflunomide or a pharmaceutically acceptable salt thereof; and
b) monitoring the blood pressure of the patient during the treatment.

The method of managing the risk of high blood pressure can further comprise a step of administering to the patient a drug which is known to be effective for treating hypertension, when hypertension is diagnosed during the treatment.

Another embodiment of the invention is related to a method for treating multiple sclerosis in a patient having multiple sclerosis and an active acute or chronic infection in need thereof, comprising:
a) resolving the acute or chronic infection prior to the initiation of the treatment; and
b) then administering to the patient a pharmaceutically effective amount of teriflunomide or a pharmaceutically acceptable salt thereof.

Another embodiment of the invention is related to a method of managing the risk of a serious infection to allow an effective and safe use of teriflunomide or a pharmaceutically acceptable salt thereof in the treatment of multiple sclerosis in a patient in need thereof comprising:
a) administering to the patient a pharmaceutically effective amount of teriflunomide or a pharmaceutically acceptable salt thereof;
b) if the patient develops a serious infection during the treatment, then considering suspending the treatment; and
c) if the treatment is suspended, reassessing the benefits and risks prior to re-initiation of the treatment.

Another embodiment of the invention is related to a method of managing the risk of a bone marrow disorder to allow an effective and safe use of teriflunomide or a pharmaceutically acceptable salt thereof in the treatment of multiple sclerosis in a patient in need thereof comprising:
a) obtaining a complete blood count (CBC) of the patient wherein the complete blood count is measured with six month prior to the treatment;
b) administering to the patient a pharmaceutically effective amount of teriflunomide or a pharmaceutically acceptable salt thereof;
c) monitoring CBC of the patient during the treatment.

The method of managing the risk of a bone marrow disorder can further comprise a step that if neutropenia is diagnosed with the patient during the treatment with teriflunomide, the treatment should be considered to be suspended and optionally reinitiating the treatment after the CBC of the patient is back to normal level.

Another embodiment of the invention is related to a method of managing the risk of polyneuropathy to allow an effective and safe use of teriflunomide or a pharmaceutically acceptable salt thereof in the treatment of multiple sclerosis in a patient in need thereof comprising:
a) administering to the patient a pharmaceutically effective amount of teriflunomide or a pharmaceutically acceptable salt thereof; and
b) if polyneuropathy is diagnosed during the treatment, then the treatment should be discontinued.

A variety of educational materials may be employed to ensure proper prescribing, dispensing and patient compliance according the methods described herein. For example, a variety of literature and other material, such as prescribing information, package inserts, medication guides, physician sheets, health care professional information sheets, medical journal advertisement, and product website may describe the risks and benefits of taking teriflunomide.

Another embodiment of the invention is related to an article of manufacture comprising:
a) a packaging material;
b) teriflunomide or a pharmaceutically acceptable salt thereof; and
c) a label or package insert contained within the packaging material indicating that liver enzymes should be monitored during the treatment with teriflunomide.

Another embodiment of the invention is related to an article of manufacture comprising:
d) a packaging material;
e) teriflunomide or a pharmaceutically acceptable salt thereof; and
f) a label or package insert contained within the packaging material indicating that if significant liver injury is confirmed or if the elevation of liver transaminases exceeds three times the upper limit of normal, then the treatment with teriflunomide should be discontinue.

Another embodiment of the invention is related to an article of manufacture comprising:
a) a packaging material;
b) teriflunomide or a pharmaceutically acceptable salt thereof; and
c) a label or package insert contained within the packaging material indicating that blood pressure should be monitored during treatment with teriflunomide.

Another embodiment of the invention is related to an article of manufacture comprising:
a) a packaging material;
b) teriflunomide or a pharmaceutically acceptable salt thereof; and
c) a label or package insert contained within the packaging material indicating that women of childbearing potential must use effective contraception to avoid pregnancy while taking teriflunomide.

Another embodiment of the invention is related to an article of manufacture comprising:
a) a packaging material;
b) teriflunomide or a pharmaceutically acceptable salt thereof; and
c) a label or package insert contained within the packaging material indicating that patients with an active acute or chronic infection should not start treatment with teriflunomide until the infection is resolved.

Another embodiment of the invention is related to an article of manufacture comprising:
a) a packaging material;
b) teriflunomide or a pharmaceutically acceptable salt thereof; and
c) a label or package insert contained within the packaging material indicating that if the patient develops a serious infection during the treatment, then the treatment should be suspended and the benefits and risks should be assessed prior to re-initiation of the treatment.

Another embodiment of the invention is related to an article of manufacture comprising:
a) a packaging material;
b) teriflunomide or a pharmaceutically acceptable salt thereof; and
c) a label or package insert contained within the packaging material indicating that complete blood count should be periodically monitored during treatment with teriflunomide.

Another embodiment of the invention is related to an article of manufacture comprising:
a) a packaging material;
b) teriflunomide or a pharmaceutically acceptable salt thereof; and
c) a label or package insert contained within the packaging material indicating that if the diagnosis of polyneuropathy is confirmed, the treatment with teriflunomide should be discontinued.

Another embodiment of the invention is related to an article of manufacture comprising:
a) a packaging material;
b) teriflunomide or a pharmaceutically acceptable salt thereof; and
c) a label or package insert contained within the packaging material indicating that teriflunomide should not be given to a patient who is hypersensitive to teriflunomide.

Another embodiment of the invention is related to a package comprising teriflunomide or a pharmaceutically acceptable salt thereof, and a label, wherein the label comprises one or more messages selected from:
a) liver enzymes should be monitored during the treatment with teriflunomide;
b) if significant liver injury is confirmed or if the elevation of liver transaminases exceeds three times the upper limit of normal, then the treatment with teriflunomide should be discontinue;
c) blood pressure should be monitored during treatment with teriflunomide;
d) women of childbearing potential must use effective contraception to avoid pregnancy while taking teriflunomide;
e) patients with an active acute or chronic infection should not start treatment with teriflunomide until the infection is resolved;
f) if the patient develops a serious infection during the treatment, then the treatment should be suspended and the benefits and risks should be assessed prior to re-initiation of the treatment;
g) complete blood count should be periodically monitored during treatment with teriflunomide;
h) the diagnosis of polyneuropathy is confirmed, the treatment with teriflunomide should be discontinued; and
i) teriflunomide should not be given to a patient who is hypersensitive to teriflunomide.

Another embodiment of the invention is related to a method of promoting the use of teriflunomide or a pharmaceutically acceptable salt thereof, comprising conveying to a recipient at least one message selected from:
a) liver enzymes should be monitored during the treatment with teriflunomide;
b) if significant liver injury is confirmed or if the elevation of liver transaminases exceeds three times the upper limit of normal, then the treatment with teriflunomide should be discontinue;
c) blood pressure should be monitored during treatment with teriflunomide;
d) women of childbearing potential must use effective contraception to avoid pregnancy while taking teriflunomide;
e) patients with an active acute or chronic infection should not start treatment with teriflunomide until the infection is resolved;
f) if the patient develops a serious infection during the treatment, then the treatment should be suspended and the benefits and risks should be assessed prior to re-initiation of the treatment;
g) complete blood count should be periodically monitored during treatment with teriflunomide;
h) the diagnosis of polyneuropathy is confirmed, the treatment with teriflunomide should be discontinued; and
i) teriflunomide should not be given to a patient who is hypersensitive to teriflunomide.

Another embodiment of the invention is related to a method of providing teriflunomide or a pharmaceutically acceptable salt thereof, wherein teriflunomide or the pharmaceutically acceptable salt thereof is provided along with information indicating that:
a) liver enzymes should be monitored during the treatment with teriflunomide;
b) if significant liver injury is confirmed or if the elevation of liver transaminases exceeds three times the upper limit of normal, then the treatment with teriflunomide should be discontinue;
c) blood pressure should be monitored during treatment with teriflunomide;
d) women of childbearing potential must use effective contraception to avoid pregnancy while taking teriflunomide;
e) patients with an active acute or chronic infection should not start treatment with teriflunomide until the infection is resolved;
f) if the patient develops a serious infection during the treatment, then the treatment should be suspended and the benefits and risks should be assessed prior to re-initiation of the treatment;
g) complete blood count should be periodically monitored during treatment with teriflunomide;
h) the diagnosis of polyneuropathy is confirmed, the treatment with teriflunomide should be discontinued; and
i) teriflunomide should not be given to a patient who is hypersensitive to teriflunomide.

The present invention may be better understood by reference to the following non-limiting Example, which are exemplary of the invention. They should in no way be construed, however, as limiting the broad scope of the invention.

### EXAMPLE

TEMSO (Teriflunomide Multiple Sclerosis Oral trial) is a Phase III randomized, double-blind, placebo-controlled, parallel-group study designed to evaluate the efficacy and safety of teriflunomide in reducing the frequency of relapses and accumulation of physical disability in patients with RMS over 2 years.

### Methods

### Patients

Patients eligible for enrolment were aged 18-55 years, met McDonald's criteria for MS diagnosis, and exhibited a relapsing clinical course, with or without progression. They were required to have a score of no more than 5.5 on the Kurtzke Expanded Disability Status Scale (EDSS), a minimum of two clinical relapses in the previous 2 years or one relapse during the preceding year, but no relapses in the 60 days before randomization. During the 4 weeks prior to randomization, patients were required to be clinically stable and have signed both the informed consent form and informed consent for HIV testing.

Patients presenting with any of the following were excluded from the study.
Patients with significantly impaired bone marrow function or significant anemia, leukopenia, or thrombocytopenia:
   Hematocrit <24% and/or
   Absolute white blood cell count <4.0×10⁹/L and/or
   Platelet count <150×10⁹/L and/or
   Absolute neutrophil count 1.5×10⁹/L
Patients with a congenital or acquired severe immunodeficiency, a history of cancer (except for basal or squamous cell skin lesions which had been surgically excised with no evidence of metastasis), lymphoproliferative disease, or any patients who had received lymphoid irradiation
Known history of active tuberculosis not adequately treated
Persistent significant or severe infection
Patients who were pregnant or breastfeeding or who planned to conceive during the course of the trial; women of childbearing potential and men were required to use an acceptable method of contraception
Use of disallowed therapies including phenyloin, warfarin, tolbutamide, St John's Wort or cholestyramine in the 4 weeks prior to randomization
Treatment with adrenocorticotrophic hormone (ACTH) or systemic corticosteroids 4 weeks prior to randomization
Prior or concomitant use of cladribine, mitoxantrone, or other immunosuppressant agents such as azathioprine, cyclophosphamide, cyclosporine, methotrexate or mycophenolate
Previous treatment with teriflunomide or leflunomide
Prior use of natalizumab
Prior use of interferons or cytokine therapy in the preceding 4 months
Prior use of glatiramer acetate therapy in the preceding 6 months
Prior use of intravenous immunoglobulins in the preceding 6 months
Prior use of any investigational drug in the preceding 6 months
Patients likely to require treatment during the study period with drugs not permitted by the clinical study protocol
Contraindication for MRI including presence of pacemaker, metallic implants in high-risk areas (e.g. artificial heart valves, aneurysm/vessel clips), presence of metallic material (e.g. shrapnel) in high-risk areas, known history of allergy to any contrast medium, or history of claustrophobia that would prevent completion of all protocol-scheduled MRI visits; hip implants were not contraindicated
Patients with liver function impairment or persisting elevations of alanine aminotransferase, serum aspartate aminotransferase, or direct bilirubin greater than 1.5-times the upper limit of normal (ULN)
Persisting elevations of serum amylase or lipase greater than 2×ULN
Known history of active hepatitis
Hypoproteinemia (e.g. in case of severe liver disease or nephrotic syndrome) with serum albumin <3.0 g/dL
Known history of chronic pancreatic disease or pancreatitis
Moderate-to-severe impairment of renal function, as shown by serum creatinine >1.5 mg/dL
Clinically relevant cardiovascular, hepatic, neurologic, endocrine, or other major systemic disease making implementation of the protocol or interpretation of the study results difficult or that would put the subject at risk by participating in the study
History of drug or alcohol abuse
Psychiatric condition rendering the subject unable to understand the nature, scope, and possible consequences of the study
Patients who were unlikely to comply with protocol (e.g. uncooperative attitude, inability to return for follow-up visits), and known unlikelihood of completing the study
Patient was the investigator or any sub-investigator, research assistant, pharmacist, study coordinator, other staff or relative thereof directly involved in the conduct of the protocol
Human immunodeficiency virus (HIV)-positive subject
Patients were not allowed to be randomized into the study more than once

### Study Design and Randomization

Following a screening phase of up to 4 weeks, eligible patients were randomized (1:1:1) to receive a once-daily oral dose of placebo or teriflunomide, 7 mg or 14 mg, for 108 weeks. Randomization was stratified by baseline EDSS score (≦3.5 and >3.5) and by trial site. Participants successfully completing the study were eligible to enter a long-term extension.

### Study Procedures

A treatment neurologist at each site was responsible for evaluating patient eligibility, supervision of study medication administration, recording and treating adverse events and assessing relapses, and monitoring safety assessments. An independent examining neurologist, blinded to treatment assignment, performed all EDSS scores and functional system assessments. EDSS scores were determined at screening, baseline, every 12 weeks following baseline visit, and at unscheduled relapse visits. MRI scans were performed at baseline and Weeks 24, 48, 72 and 108, and conducted according to the study protocol; image data were collected at clinical site MRI facilities.

For suspected relapses occurring between study visits, patients were required to attend the study site within 7 days of symptom onset for objective assessment by the examining neurologist. Relapses could be treated with intravenous corticosteroids at the investigator's discretion.

Safety was evaluated by the occurrence of adverse events reported by the patient or the investigator. Laboratory tests were performed during screening, at baseline, and then every 2 weeks for the first 24 weeks, then every 6 weeks until study completion. Physical and neurologic examinations were performed at Week 12 and then every 24 weeks. An abdominal ultrasound to assess pancreatic abnormalities was done prior to the study and then every 24 weeks. Patients who withdrew from the study or did not participate in the extension underwent an 11-day washout procedure with cholestyramine or activated charcoal.

### Study Endpoints

The primary study objective was to determine the effect of teriflunomide in reducing annualized relapse rate (ARR; the number of confirmed relapses per patient-year). A relapse was defined as the appearance of a new clinical sign or symptom or clinical worsening of a previous sign or symptom that had been stable for at least 30 days and persisted for a minimum of 24 hours in the absence of fever. Confirmed relapses required an increase of 1 point in at least two functional systems, or an increase of 2 points in at least one functional system (excluding bowel/bladder and cerebral function), or an increase of 0.5 points in EDSS score (1.0 for EDSS=0) from the previous clinically stable assessment.

The key secondary endpoint was to determine the effect of teriflunomide on delaying disability progression at Week 108 as assessed by changes in EDSS. Sustained disability progression was defined as a persisting increase from baseline of at least 1.0 point on the EDSS (or at least 0.5 for patients with a baseline EDSS score greater than 5.5) for at least 12 weeks. The key prespecified MRI endpoint was the effect of teriflunomide on burden of disease (total lesion volume). Other MRI endpoints included number of gadolinium (Gd)-enhancing T1 lesions, volume of T1 hypointense lesion components (‘black holes’), number of unique active lesions and brain atrophy. Patient-reported fatigue, assessed by the Fatigue Impact Scale (FIS), was an additional secondary endpoint.

### Adverse Event Monitoring

### Hepatic and Bone Marrow Disorders

Laboratory testing of liver function and complete blood cell count with differential were performed at baseline, then every 2 weeks for the first 24 weeks of treatment and every 6 weeks thereafter. Patients with any of the following adverse events were discontinued from the study and were subsequently followed-up:
Any confirmed increase (repeated value within 48 hours) in alanine aminotransferase (≧3×ULN) with or without increase in total bilirubin level
Any occurrence of confirmed neutrophil count less than 1.0×10⁹ cells/L with or without infection
Any potentially life-threatening cytopenias detected during the follow-up of laboratory abnormalities as per the investigator's judgment

### Pulmonary Disorders

Pulmonary symptoms suggestive of pulmonary toxicity (e.g. cough, dyspnoea) were assessed by a pulmonologist using appropriate diagnostic testing.

### Peripheral Neuropathy

Neurologic symptoms suggestive of a peripheral neuropathy (e.g. bilateral numbness, tingling of the feet or hands) were followed up with nerve conduction studies.

### Pancreatic Disorders

Laboratory testing of pancreatic enzymes was performed at baseline, then every 2 weeks for the first 24 weeks of treatment and every 6 weeks thereafter. An abdominal ultrasound was performed at baseline and then every 24 weeks until study completion. Any suspected abnormal ultrasound was followed up with a CT scan with contrast, or by MRI. Patients with any of the following adverse events were discontinued from the study and subsequently followed-up.
Any confirmed increase (repeated value within 48 hours) in blood amylase or lipase (≧5×ULN) without signs or symptoms
Any confirmed increase (repeated value within 48 hours) in blood amylase or lipase (≧2×ULN) with signs or symptoms consistent with pancreatitis
Any patient with a CT-scan or MRI lesion suggestive of pancreatic abnormality was discontinued from treatment.

### Pregnancy Testing

Serum pregnancy testing was performed at baseline, Week 10, Week 24 and then every 12 weeks thereafter. If the pregnancy test was positive, the study medication was discontinued, and the physician was required to inform the patient of the risk of pregnancy and to discuss the potential alternatives. If the patient decided to continue with their pregnancy, a washout was required. If the patient decided not to continue with their pregnancy, the study medication could be resumed 2 weeks after termination of the pregnancy.

### Statistical Analysis

A sample size of 360 randomized patients per group was required to provide 95% statistical power to detect relative risk reductions (RRR) of 25% in ARR after 2 years, assuming a placebo ARR of 0.74 and standard deviation of 0.626. All analyses followed an intention-to-treat (ITT) principle and all inferential analyses were performed at the 2-sided 5% level of significance. ARR was analyzed using a Poisson regression model with robust error variance. The model included the total number of confirmed relapses as the response variable, and treatment, baseline EDSS strata, and region as covariates, and log transformed duration of treatment as offset variable.

Disability progression was analyzed using a log-rank test with time to first disability progression as the dependent variable, treatment as test variable, and region and baseline EDSS score as strata variables. Kaplan-Meier estimates and curves of the cumulative incidence were used to assess the rate of disability progression in patients across time points.

The ITT population comprised randomized patients exposed to at least 1 day of study medication.

A step-down testing procedure was used to analyze ARR and time to disability progression; the two doses of teriflunomide were compared sequentially with placebo.

Change from baseline in burden of disease and T1 hypointense lesion volume were analysed with a mixed-effect model with repeated measures (MMRM) using cubic root transformed data. Atrophy was analysed as change from baseline brain volume with the MMRM. The number of Gd-enhancing T1 lesions and number of unique active lesions per scan were analysed using a Poisson regression model with robust error variance. The percentage of patients free from lesions was analysed using a logistic regression model. MRI scans obtained within 14 days of systemic corticosteroid therapy were excluded from all analyses.

All subjects were analyzed according to the treatment to which they were randomized. The ITT population was used for the analyses of all efficacy variables. The per-protocol population comprised the ITT population, excluding subjects with major efficacy-related protocol deviations and was used for the analysis of the primary efficacy variable and key secondary variables.

To control type 1 error rate, a step-down testing procedure was used that compared teriflunomide with placebo in the following order: ARR, first in association with 14 mg teriflunomide, and next in association with 7 mg teriflunomide, followed by time to disability progression, first with 14 mg teriflunomide and next with 7 mg teriflunomide. Each test was performed only if the preceding evaluation was significant at the 5% level.

The MMRM model (mixed-effect model with repeated measures) included treatment, EDSS strata, region, visit, treatment-by-visit interaction, baseline value and baseline-by-visit interaction as covariates. The percentage of patients free from lesions was analysed by a logistic regression model that included treatment, number of lesions at baseline, EDSS strata and region as covariates. The Poisson regression model included treatment, number of lesions at baseline, EDSS strata and region as covariates, and log-transformed number of scans as an offset variable. The change from baseline in fatigue impact scale (FIS) total score was analyzed using MMRM.

### Results

### Study Population

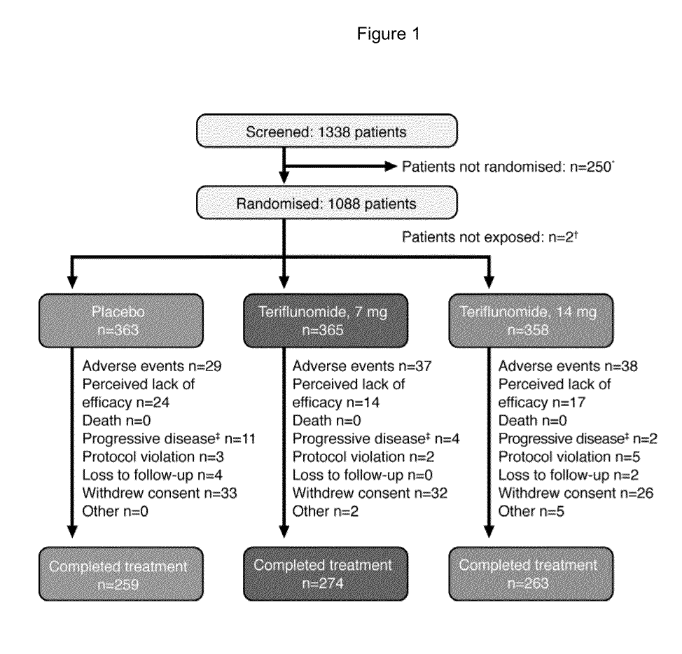

From 24 Sep. 2004 to 13 Mar. 2008, patients were recruited from 144 clinical centers across 21 countries. A total of 1,088 patients were randomized to treatment; 1,086 (99.8%) were exposed to study treatment and formed the ITT population. A total of 796 (73.2%) patients completed study treatment with similar proportions of patients in the three treatment groups (71.3%, 74.9%, and 73.3% in the placebo, 7 mg and 14 mg groups, respectively; FIG. 1). A lower number of patients discontinued treatment due to disease progression in the 14 mg teriflunomide group compared with placebo (p=0.02; FIG. 1).

Baseline demographics and disease characteristics are shown in Table 1. Although the majority of patients had relapsing remitting MS, a subset of patients were diagnosed with secondary progressive and progressive relapsing MS. The overall MRI burden of disease was approximately 19 mL, with 36.2% having enhancement(s) at baseline. Seventy-three percent of patients had not received any disease-modifying therapy (DMT) treatment during the 2 years prior to study entry.

### Efficacy

Relapses

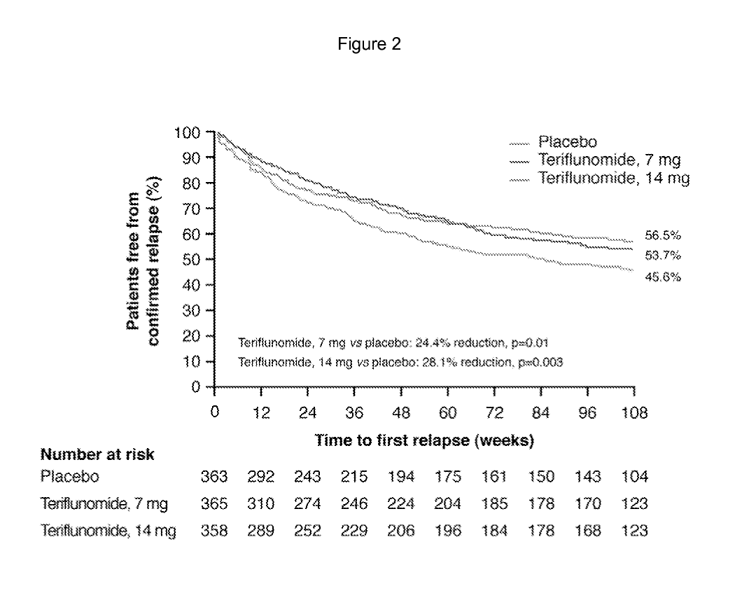

Teriflunomide significantly reduced ARR (0.539, 0.370 and 0.369, for placebo, teriflunomide 7 mg and 14 mg), representing relative risk reduction (RRR) of 31.2% and 31.5%, respectively; p<0.001 for both doses vs placebo (Table 2). In both teriflunomide groups, the time to first relapse was longer, the risk of relapse was reduced, and more patients remained free of relapse compared with the placebo group (Table 2 and FIG. 2).

### Disability

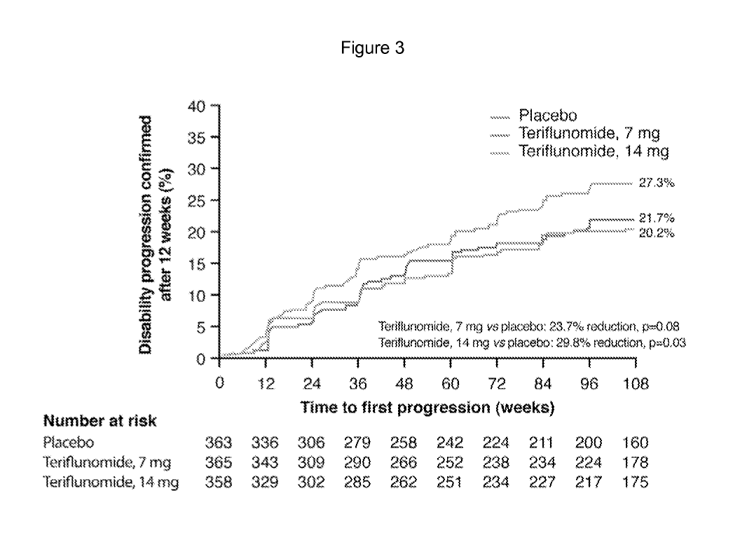

The estimated proportion of patients with 12-week-confirmed disability progression was 27.3%, 21.7% and 20.2% in the placebo, 7 mg, and 14 mg groups, respectively, representing RRRs of 23.7% (p=0.08) and 29.8% (p=0.03) compared with placebo (Table 2 and FIG. 3).

### MRI Outcomes

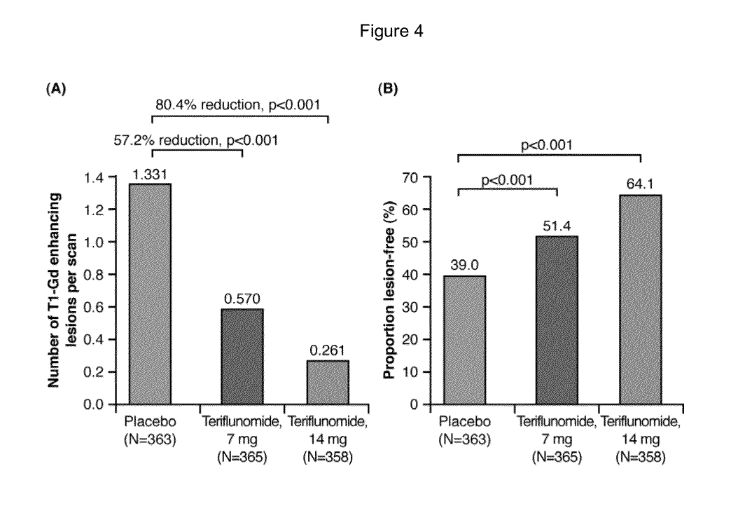

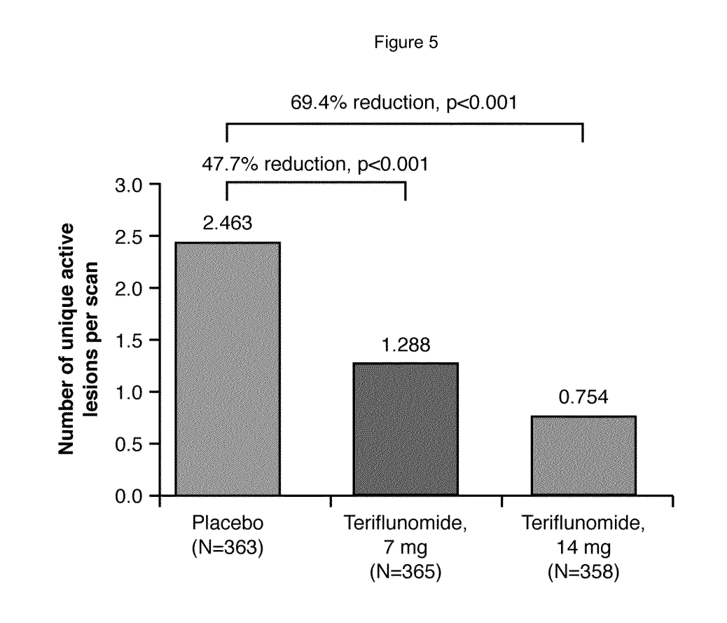

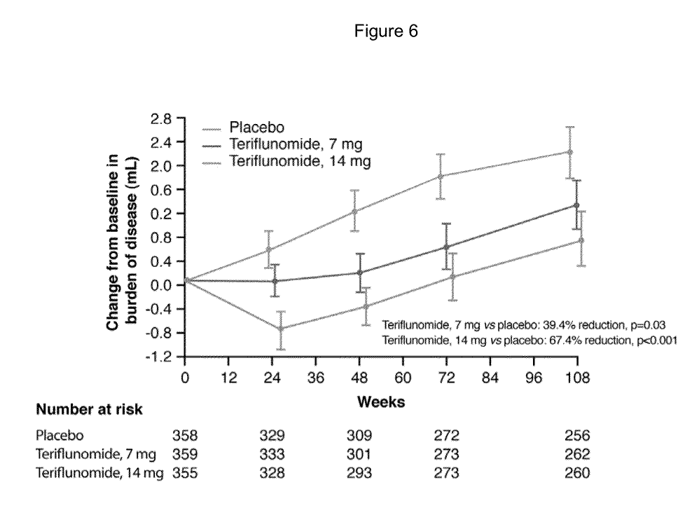

Both teriflunomide doses improved several MRI parameters of disease activity compared with placebo (Table 2). Change from baseline in burden of disease was significantly lower in both teriflunomide groups compared with placebo (p=0.03 and p<0.001, respectively). The relative increase in burden of disease was reduced by 67% in the 14 mg group. Both teriflunomide groups had significantly fewer Gd-enhancing T1 lesions per scan than those in the placebo group (RRRs vs placebo of 57.2% and 80.4%; p<0.001 for both doses), with more teriflunomide-treated patients free from Gd-enhancing lesions (Table 2 and FIG. 4). Fewer unique active lesions per scan were observed in both teriflunomide groups compared with placebo (RRR vs placebo of 47.7% and 69.4%; p<0.001 for both doses) (Table 2 and FIG. 5). The change from baseline in T1 hypointense lesion volume was reduced by 31.3% in the 14 mg group compared with placebo (p=0.02). No significant differences were observed in brain atrophy from baseline across the three groups (Table 2).

### Impact on Fatigue

Patient-reported fatigue remained stable during the study in all treatment groups, with only small changes from baseline in FIS scores (Table 2).

### Safety and Tolerability

### Common Treatment Emergent Adverse Events

Teriflunomide was well-tolerated with similar numbers of TEAEs (87.5%, 89.1%, 90.8%), serious TEAEs (12.8%, 14.1%, 15.9%), and TEAEs leading to treatment discontinuation (8.1%, 9.8%, 10.9%) in the placebo, 7 mg, and 14 mg teriflunomide groups, respectively (Table 3).

Among the most common TEAEs (≧10% crude incidence) with an increased incidence in the teriflunomide groups compared with placebo and with a dose-effect were diarrhea, nausea, hair thinning/decreased hair density, and ALT increase (reported as an adverse event). These events rarely led to treatment discontinuation (diarrhea: 0.0%, 0.3% and 0.3%; nausea: 0.0%, 0.3% and 0.0%; hair thinning/decreased hair density: 0.0%, 0.5% and 1.4%; for placebo, 7 mg, and 14 mg teriflunomide groups, respectively). No deaths occurred.

### Hepatic Disorders

Although the incidence of ALT increase (≧1×ULN) was higher with teriflunomide treatment (35.9%, 54.0% and 57.3%), the incidence of ALT ≧3×ULN was similar across treatment groups (6.7%, 6.3%, and 6.7%), for placebo, teriflunomide 7 mg and 14 mg, respectively. Three patients (one in each treatment group) had increased ALT ≧3×ULN and total bilirubin ≧2×ULN; all cases were considered as non drug-induced liver injuries with alternative explanations provided (Hepatitis C, Gallbladder problems and CMV infection, respectively). A similar incidence of serious hepatic disorders (ALT >8×ULN and/or ALT >3×ULN with total bilirubin >3 mg/dL and/or any hepatic event with seriousness criterion) occurred in all groups (2.5%, 1.9%, and 2.5%, for placebo, teriflunomide 7 mg and 14 mg, respectively).

### White Blood Cell Counts

Reductions in neutrophil and lymphocyte counts (assessed by mean change from baseline) were small in magnitude (mean reductions of 1.0×10⁹/L and 0.3×10⁹/L for neutrophil counts and lymphocyte counts, respectively), were slightly more marked with the 14 mg dose, occurred over the first 3 months of treatment and stabilised over time. The proportion of patients experiencing any bone marrow TEAE was reported with a higher frequency in both teriflunomide treatment group as compared to placebo (2.2%, 9.8% and 7.5% in placebo, teriflunomide 7 mg and teriflunomide 14 mg respectively). They were mainly neutropenia diorders (0.3%, 2.2% and 4.5%). Three cases of moderate neutropenia (reduction in neutrophil count to 0.9×10⁹/L) occurred on teriflunomide; two resolved spontaneously with continued treatment and one recovered following discontinuation of the drug.

### Infections

A low and similar incidence of serious infections was reported across groups (2.2%, 1.6% and 2.5% in the placebo, 7 mg, and 14 mg groups, respectively) with no serious opportunistic infections observed. The incidence of infections and infections leading to treatment discontinuation was similar across all groups.

### Pregnancies

Eleven pregnancies occurred in female patients during the trial; four spontaneous abortions (1 placebo and 3 in 14 mg group) and six induced abortions (5 in 7 mg and 1 in 14 mg group). One patient in the 14 mg group (treated for 31 days of pregnancy) delivered a healthy baby with no reported health concerns after 2 years. All patients discontinued study treatment and underwent an 11-day washout procedure as soon as they were made aware of their pregnancy.

### Malignancies

Malignant neoplasms were reported in four patients; three in the placebo group (1 breast cancer, 1 thyroid cancer and 1 uterine cervix cancer) and one in the teriflunomide 14 mg group (cervix carcinoma in situ (stage 0) reported after 1.5 years treatment, which recovered following local surgery).

### Blood Pressure Increase

The proportion of patients with TEAEs related to blood pressure increase was higher in the teriflunomide groups compared with the placebo group (3.1%, 5.4%, and 5.0% in the placebo, teriflunomide 7 mg and 14 mg groups, respectively). The average increase in systolic blood pressure was <4 mmHg and diastolic blood pressure was <2 mmHg, respectively. No patient discontinued treatment due to increased blood pressure.

### Pancreatic Disorders

No increases in amylase and lipase (assessed by mean change from baseline) were reported across the three treatment groups. The only case of pancreatitis was in a placebo patient.

### Hypersensitivity/Skin Reactions

Based on selected SMQs terms, the proportion of patients with treatment-emergent adverse events (TEAEs) potentially related to hypersensitivity/skin disorders was generally higher in the teriflunomide groups compared with placebo (7.2%, 10.3%, and 11.2%, for the placebo, teriflunomide 7 mg and 14 mg groups, respectively). Urticaria, erythema, pruritus and pruritic rash were observed with low incidences, but were globally more frequent in the teriflunomide treatment groups compared with placebo (Table 5). One patient in the 14 mg group discontinued treatment due to pruritus.

### Peripheral Neuropathy

Ten patients receiving teriflunomide treatment experienced mild or moderate peripheral neuropathy with conduction confirmation (four in the 7 mg group and six in the 14 mg group vs none in the placebo group). However, five of these cases were described as mononeuropathy and three patients recovered while on treatment. Treatment was discontinued for two patients in one center (one of these patients had recovered from this AE at the time of discontinuation). Polyneuropathy was reported with very low incidencies, but more frequently with teriflunomide treatment than with placebo (0%, 0.3% and 0.8% in placebo, teriflunomide 7 mg and teriflunomide 14 mg, respectively).

### Pulmonary Disorders (Interstitial Lung Disease)

The only case of pneumonitis was reported in the placebo group. No adverse event potentially attributed to interstitial lung disease (based on SMQ terms) was reported in the teriflunomide groups.

### Additional Details on Cases of Neutrophil Decrease

Two cases of moderate neutrophil count decrease (classed as serious AEs) were reported in the teriflunomide 14 mg group; reductions of 0.95×10⁹/L and 0.90×10⁹/L, with values returning to 2.09×10⁹/L and 3.82×10⁹/L, respectively, a few days later, with teriflunomide treatment continuing for both patients. One case of neutropenia led to treatment discontinuation from the 7 mg group; neutrophil counts decreased to 0.87×10⁹/L and returned to 1.34×10⁹/L, three days after treatment discontinuation.

### CONCLUSION

These findings show that teriflunomide is a safe and effective new oral monotherapy for RMS and represents a potential first-line treatment option in this patient population.

TABLE 1
Patient demographics and baseline clinical and MRI variables

| | | | Teriflunomide | | | | | |
|---|---|---|---|---|---|---|---|---|
| | Placebo | | 7 mg | | 14 mg | | | |
| | (N = 363) | | (N = 366) | | (N = 359) | | All (N = 1088) | |
| Baseline demographics | | | | | | | | |
| Age - years | | | | | | | | |
| Mean (SD) | 38.4 | (9.0) | 37.4 | (9.0) | 37.8 | (8.2) | 37.9 | (8.8) |
| Median (range) | 39.0 | (18-55) | 38.5 | (18-55) | 38.0 | (18-55) | 38.0 | (18-55) |
| Female sex - n (%) | 275 | (75.8) | 255 | (69.7) | 255 | (71.0) | 785 | (72.2) |
| Race - n (%) | | | | | | | | |
| Caucasian | 356 | (98.3) | 355 | (97.3) | 347 | (96.9) | 1058 | (97.5) |
| Region - n (%) | | | | | | | | |
| Western Europe | 167 | (46.0) | 167 | (45.6) | 170 | (47.4) | 504 | (46.3) |
| Eastern Europe | 114 | (31.4) | 116 | (31.7) | 108 | (30.1) | 338 | (31.1) |
| Americas | 82 | (22.6) | 83 | (22.7) | 81 | (22.6) | 246 | (22.6) |
| Baseline disease | | | | | | | | |
| characteristics | | | | | | | | |
| Time from first symptom | | | | | | | | |
| of MS - years | | | | | | | | |
| Mean (SD) | 8.6 | (7.1) | 8.8 | (6.8) | 8.7 | (6.7) | 8.7 | (6.9) |
| Median (range) | 6.3 | (0.3-35.7) | 7.0 | (0.3-32.6) | 7.2 | (0.4-31.6) | 6.8 | (0.3-35.7) |
| Relapses | | | | | | | | |
| Within previous year | | | | | | | | |
| Mean (SD) | 1.4 | (0.7) | 1.4 | (0.7) | 1.3 | (0.7) | 1.4 | (0.7) |
| Median (range) | 1.0 | (0-6) | 1.0 | (0-6) | 1.0 | (0-4) | 1.0 | (0-6) |
| Within previous 2 | | | | | | | | |
| years | | | | | | | | |
| Mean (SD) | 2.2 | (1.0) | 2.3 | (1.2) | 2.2 | (1.0) | 2.2 | (1.1) |
| Median (range) | 2.0 | (1-7) | 2.0 | (1-12) | 2.0 | (1-9) | 2.0 | (1-12) |
| MS subtype - n (%) | | | | | | | | |
| Relapsing remitting | 329 | (90.6) | 333 | (91.0) | 333 | (92.8) | 995 | (91.5) |
| Secondary | 22 | (6.1) | 17 | (4.6) | 12 | (3.3) | 51 | (4.7) |
| progressive | | | | | | | | |
| Progressive relapsing | 12 | (3.3) | 16 | (4.4) | 14 | (3.9) | 42 | (3.9) |
| Previous DMT in last 2 | 90 | (24.8) | 102 | (27.9) | 102 | (28.4) | 294 | (27.0) |
| years - n (%) | | | | | | | | |
| EDSS score | | | | | | | | |
| Mean (SD) | 2.68 | (1.34) | 2.68 | (1.34) | 2.67 | (1.24) | 2.68 | (1.31) |
| Median (range) | 2.5 | (0.0-6.0) | 2.5 | (0.0-6.0) | 2.5 | (0.0-5.5) | 2.5 | (0.0-6.0) |
| FIS score | | | | | | | | |
| Mean (SD) | 53.2 | (37.9) | 50.4 | (35.6) | 50.3 | (35.9) | 51.3 | (36.5) |
| Median (range) | 49.0 | (0.0-152.0) | 47.1 | (0.0-141.1) | 46.0 | (0.0-143.0) | 47.0 | (0.0-152.0) |
| Baseline MRI | | | | | | | | |
| characteristics | | | | | | | | |
| Burden of disease (mL)* | | | | | | | | |
| Mean (SD) | 19.34 | (18.94) | 20.37 | (20.59) | 18.08 | (17.49) | 19.27 | (19.06) |
| Median (range) | 12.75 | (0.1-83.7) | 13.96 | (0.2-146.3) | 12.39 | (0.3-88.8) | 13.02 | (0.1-146.3) |
| Patients with | 137 | (38.2) | 127 | (35.3) | 125 | (35.2) | 389 | (36.2%) |
| gadolinium-enhancing | | | | | | | | |
| lesions - n (%) | | | | | | | | |
| Number of gadolinium- | | | | | | | | |
| enhancing lesions on | | | | | | | | |
| T1-weighted images | | | | | | | | |
| Mean (SD) | 1.66 | (3.55) | 1.50 | (3.96) | 1.81 | (5.17) | 1.66 | (4.28) |
| Median (range) | 0 | (0.0-26.0) | 0 | (0.0-38.0) | 0 | (0.0-50.0) | 0 | (0.0-50.0) |
| Volume of hypointense | | | | | | | | |
| lesions on T1-weighted | | | | | | | | |
| images - mL | | | | | | | | |
| Mean (SD) | 3.26 | (3.64) | 3.35 | (3.96) | 2.91 | (3.25) | 3.17 | (3.63) |
| Median (range) | 1.87 | (0.02-20.14) | 2.03 | (0.00-23.67) | 1.76 | (0.01-29.10) | 1.88 | (0.00-29.10) |
| Brain parenchymal | | | | | | | | |
| fraction | | | | | | | | |
| Mean (SD) | 0.76 | (0.02) | 0.76 | (0.02) | 0.76 | (0.02) | 0.76 | (0.02) |
| Median (range) | 0.76 | (0.71-0.84) | 0.76 | (0.71-0.84) | 0.76 | (0.72-0.84) | 0.76 | (0.71-0.84) |

Randomized population
*Burden of disease is defined as the total volumes of hyperintense T2 lesion component plus hypointense T1 lesion component on MRI.

TABLE 2
Clinical and MRI results

| | Placebo | | 7 mg | | 14 mg | |
|---|---|---|---|---|---|---|
| Variable | (N = 363) | | (N = 365) | | (N = 358) | |
| Relapses | | | | | | |
| Adjusted Annualized Relapse Rate (95% | 0.539 | (0.466-0.623) | 0.370 | (0.318-0.432) | 0.369 | (0.308-0.441) |
| CI)* | | | | | | |
| p-value vs placebo | | | <0.001 | | <0.001 | |
| Relative Reduction in Annualized | | | | | | |
| Relapse rate vs placebo (%) | | | 31.2 | | 31.5 | |
| Patients without relapse during 108 | 45.6 | (40.2-51.0) | 53.7 | (48.3-59.1) | 56.5 | (51.0-62.0) |
| weeks - percent (95% CI)^{†} | | | | | | |
| Hazard ratio for teriflunomide vs placebo | | | 0.756 | (0.611-0.937) | 0.719 | (0.577-0.895) |
| (95% CI)^{†‡} | | | | | | |
| p-value vs placebo | | | 0.01 | | 0.003 | |
| No. of relapses - no. of patients (%) | | | | | | |
| 0 | 179 | (49.3) | 211 | (57.8) | 217 | (60.6) |
| 1 | 97 | (26.7) | 92 | (25.2) | 86 | (24.0) |
| 2 | 48 | (13.2) | 49 | (13.4) | 33 | (9.2) |
| 3 | 22 | (6.1) | 10 | (2.7) | 16 | (4.5) |
| ≧4 | 17 | (4.7) | 3 | (0.8) | 6 | (1.7) |
| Disability | | | | | | |
| Patients with a 12-week sustained | 27.3 | (22.3-32.3) | 21.7 | (17.1-26.3) | 20.2 | (15.6-24.7) |
| disability progression - percent (95% | | | | | | |
| CI)^{†} | | | | | | |
| Hazard ratio for teriflunomide vs placebo | | | 0.763 | (0.555-1.049) | 0.702 | (0.506-0.973) |
| (95% CI)^{‡} | | | | | | |
| Hazard ratio reduction vs placebo (%) | | | 23.7 | | 29.8 | |
| p-value vs placebo | | | 0.08 | | 0.03 | |
| Fatigue Impact scale score^{¶} | | | | | | |
| LS mean (SE) change from baseline | 4.3 | (1.7) | 2.3 | (1.6) | 3.8 | (1.7) |
| p-value vs placebo | | | 0.39 | | 0.83 | |
| MRI outcome | | | | | | |
| Burden of disease (mL)^{§}** | | | | | | |
| Mean (SD) change from baseline | 2.208 | (7.002) | 1.308 | (6.799) | 0.723 | (7.591) |
| LS mean (SE) change from baseline^{§} | 0.132 | (0.018) | 0.080 | (0.018) | 0.043 | (0.018) |
| LS mean (SE) difference vs placebo^{§} | | | −0.053 | (0.024) | −0.089 | (0.025) |
| Relative reduction vs placebo (%)^{§} | | | 39.4 | | 67.4 | |
| p-value vs placebo | | | 0.03 | | <0.001 | |
| Number of Gd-enhancing T1 lesions per | | | | | | |
| scan^{\|\|} | | | | | | |
| Estimate (95% CI) | 1.331 | (1.059-1.673) | 0.570 | (0.434-0.748) | 0.261 | (0.167-0.407) |
| Relative risk (95% CI) | — | | 0.428 | (0.310-0.592) | 0.196 | (0.120-0.321) |
| Relative reduction vs placebo (%) | | | 57.2 | | 80.4 | |
| p-value vs placebo | | | <0.001 | | <0.001 | |
| Proportion of patients free from Gd- | 135 | (39.0) | 180 | (51.4) | 218 | (64.1) |
| enhancing T1 lesions n (%) | | | | | | |
| p-value vs placebo | | | <0.001 | | <0.001 | |
| Number of unique active lesions | | | | | | |
| per scan^{\|\|} | | | | | | |
| Estimate (95% CI) | 2.463 | (2.102-2.886) | 1.288 | (1.075-1.543) | 0.754 | (0.576-0.987) |
| Relative risk (95% CI) | — | | 0.523 | (0.420-0.650) | 0.306 | (0.228-0.411) |
| Relative reduction vs placebo (%) | | | 47.7 | | 69.4 | |
| p-value vs placebo | | | <0.001 | | <0.001 | |
| T1 hypointense lesion volume (mL) | | | | | | |
| Mean (SD) change from baseline | 0.533 | (1.063) | 0.499 | (1.154) | 0.331 | (1.012) |
| LS mean (SE) change from baseline^{§} | 0.096 | (0.009) | 0.079 | (0.009) | 0.066 | (0.009) |
| LS mean (SE) difference vs placebo^{§} | | | −0.016 | (0.012) | −0.030 | (0.013) |
| Relative reduction vs placebo (%)^{§} | | | 16.7 | | 31.3 | |
| p-value vs placebo | | | 0.19 | | 0.02 | |
| Brain parenchymal fraction^{¶} | | | | | | |
| LS mean (SE) change from baseline | −0.004 | (0.001) | −0.003 | (0.001) | −0.003 | (0.001) |

| | | | | | |
|---|---|---|---|---|---|
| LS mean (SE) difference vs placebo | — | 0.001 | (0.001) | 0.001 | (0.001) |

| | | |
|---|---|---|
| Relative reduction vs placebo (%) | 25.0 | 25.0 |
| p-value vs placebo | 0.19 | 0.35 |

Data presented for ITT population
*Derived from an analysis of the number of relapses with the use of a Poisson regression model adjusted for treatment, Expanded Disability Status Scale (EDSS) score strata at baseline and region, and the log of time on treatment as an offset variable
^{†}Derived from Kaplan-Meier estimate
^{‡}The hazard ratio and 95% confidence intervals were based on a Cox proportional hazard model adjusted for treatment, EDSS score strata at baseline and region. P-values were based on a log-rank test adjusted for treatment, EDSS score strata at baseline and region
^{§}MMRM analysis using cubic root transformed volume data
^{||}Poisson regression model adjusted for treatment, EDSS strata at baseline, region and baseline number of lesions, and the log of number of MRI scans as an offset variable
^{¶}MMRM analysis
**Burden of disease is defined as the total volumes of hyperintense on T2 lesion component plus hypointense on T1 lesion component as measured by MRI scan.
CI, confidence intervals;
Gd, gadolinium;
LS, least square;
SE, standard error;
ITT, intention to treat;
MMRM, mixed-effect model with repeated measures

TABLE 3
Adverse events

| | Placebo | 7 mg | 14 mg |
|---|---|---|---|
| Adverse event* | (N = 360) | (N = 368) | (N = 358) |
| All events - n (%) | | | |
| At least one treatment | 315 (87.5) | 328 (89.1) | 325 (90.8) |
| emergent adverse event | | | |
| Any treatment emergent | 29 (8.1) | 36 (9.8) | 39 (10.9) |
| adverse event leading to | | | |
| discontinuation of study drug | | | |
| Any treatment emergent | 46 (12.8) | 52 (14.1) | 57 (15.9) |
| serious adverse event | | | |
| Any event leading to death | 0 | 0 | 0 |
| Most common treatment | | | |
| emergent adverse events - no. | | | |
| of patients (%) | | | |
| Nasopharyngitis | 98 (27.2) | 94 (25.5) | 93 (26.0) |
| Headache | 64 (17.8) | 81 (22.0) | 67 (18.7) |
| Diarrhea^{‡} | 32 (8.9) | 54 (14.7) | 64 (17.9) |
| Fatigue | 51 (14.2) | 47 (12.8) | 52 (14.5) |
| Alanine aminotransferase | 24 (6.7) | 44 (12.0) | 51 (14.2) |
| increased^{§‡} | | | |
| Nausea^{‡} | 26 (7.2) | 33 (9.0) | 49 (13.7) |
| Hair thinning/decreased hair | 12 (3.3) | 38 (10.3) | 47 (13.1) |
| density^{†‡} | | | |
| Influenza | 36 (10.0) | 34 (9.2) | 43 (12.0) |
| Back pain | 47 (13.1) | 39 (10.6) | 41 (11.5) |
| Urinary tract infection | 35 (9.7) | 27 (7.3) | 37 (10.3) |
| Pain in extremity^{\|\|} | 47 (13.1) | 26 (7.1) | 33 (9.2) |

Data presented for safety population
*Treatment-emergent adverse events (TEAEs) by Medical Dictionary for Regulatory Activities (MedDRA) preferred term ≧10% in any group, ranked by decreasing order in the teriflunomide 14 mg group
^{†}MedDRA preferred term: alopecia
^{‡}TEAEs occurring at a higher rate in the teriflunomide groups
^{§}Increases reported as adverse events by the investigator; as these reports are based on investigators' judgement, the percentages reported here do not exactly match the laboratory abnormalities presented in the text
^{||}TEAEs occurring at a higher rate in the placebo group

TABLE 4
Serious adverse events related to adverse events of special interest

| | | | |
|---|---|---|---|
| | | Teriflunomide, | Teriflunomide, |
| | Placebo | 7 mg | 14 mg |
| Preferred term, n (%) | (N = 360) | (N = 368) | (N = 358) |

Infections*

| | | | |
|---|---|---|---|
| Cellulitis | 1 (0.3%) | 0 | 0 |
| Urinary tract infection | 0 | 0 | 1 (0.3%) |
| enterococcal | | | |
| Erysipelas | 0 | 1 (0.3%) | 0 |
| Appendicitis | 0 | 2 (0.5%) | 0 |
| Gastroenteritis | 2 (0.6%) | 0 | 1 (0.3%) |
| Infected cyst | 0 | 1 (0.3%) | 0 |
| Lung infection | 1 (0.3%) | 0 | 0 |
| Pneumonia | 0 | 2 (0.5%) | 0 |
| Bacteremia | 0 | 0 | 1 (0.3%) |
| Pyelonephritis | 0 | 0 | 3 (0.8%) |
| Renal abscess | 0 | 0 | 1 (0.3%) |
| Urinary tract infection | 1 (0.3%) | 0 | 1 (0.3%) |
| Cytomegalovirus hepatitis | 0 | 0 | 1 (0.3%) |
| Hepatitis C | 1 (0.3%) | 0 | 0 |
| Herpes zoster | 1 (0.3%) | 0 | 0 |
| Influenza | 1 (0.3%) | 0 | 0 |

Malignant neoplasms^{†}

| | | | |
|---|---|---|---|
| Breast cancer | 1 (0.3%) | 0 | 0 |
| Thyroid cancer | 1 (0.3%) | 0 | 0 |
| Cervix carcinoma stage 0 | 1 (0.3%) | 0 | 1 (0.3%) |

Blood disorders^{‡}

| | | | |
|---|---|---|---|
| Anemia | 0 | 1 (0.3%) | 0 |
| Neutropenia | 0 | 0 | 1 (0.3%) |
| Neutrophil count decreased | 0 | 0 | 1 (0.3%) |

Pancreatic disorders^{§}

| | | | |
|---|---|---|---|
| Pancreatitis | 1 (0.3%) | 0 | 0 |
| Lipase increased | 0 | 2 (0.5%) | 0 |

Hepatic and hepatobiliary
disorders^{||}

| | | | |
|---|---|---|---|
| Liver injury | 1 (0.3%) | 1 (0.3%) | 0 |
| Hepatitis toxic | 0 | 0 | 1 (0.3%) |
| Alanine aminotransferase | 5 (1.4%) | 5 (1.4%) | 5 (1.4%) |
| increased | | | |
| Hepatic enzyme increased | 0 | 0 | 1 (0.3%) |
| Transaminases increased | 2 (0.6%) | 1 (0.3%) | 1 (0.3%) |

*All infections and infestations in the System Organ Class (SOC);
^{†}all malignancies;
^{‡}anemia (HLGT) and white blood cell disorders (HLGT) and hematology investigations (HLGT);
^{§}exocrine pancreas conditions (HLGT) and digestive enzymes (HLT);
^{||}HLGT
Note:
no SAES related to hypersensitivity/skin reaction, peripheral neuropathy or pulmonary disorders (interstitial lung disease) were reported.
SAE, serious adverse event;
HLGT; high level general term;
HLT, high level term

TABLE 5
Incidence of TEAEs potentially related to hypersensitivity/skin reactions
based on specific SMQs

| | | | |
|---|---|---|---|
| | | Teriflunomide, | Teriflunomide, |
| | Placebo | 7 mg | 14 mg |
| Preferred term, n (%) | (N = 360) | (N = 368) | (N = 358) |

Hypersensitivity/skin reactions

| | | | |
|---|---|---|---|
| Urticaria | 2 (0.6%) | 4 (1.1%) | 3 (0.8%) |
| Erythema | 2 (0.6%) | 7 (1.9%) | 4 (1.1%) |
| Pruritus | 6 (1.7%) | 13 (3.5%) | 10 (2.8%) |
| Pruritus rash | 0 | 2 (0.5%) | 1 (0.3%) |

SMQs, standardized MedDRA queries;
MedDRA, Medical Dictionary for Regulatory Activities;
TEAEs, treatment-emergent adverse events

We claim:

1. A method for reducing annualized relapse rate in a patient having relapsing form of multiple sclerosis in need thereof, comprising administering to the patient 14 mg of teriflunomide daily, wherein the method is clinically proven effective.

2. The method according to claim 1, wherein the reduction of annualized relapse rate is about 31.5%

3. A method for delaying disability progression or reducing the risk of disability progression in a patient having relapsing form of multiple sclerosis in need thereof, comprising administering to the patient 14 mg of teriflunomide daily, wherein the method is clinically proven effective.

4. The method according to claim 3, wherein the reduction of the risk of disability progression is about 29.8%.

5. A method for managing the risk of elevation of liver enzyme to allow an effective and safe use of teriflunomide or a pharmaceutically acceptable salt thereof in the treatment of multiple sclerosis in a patient in need thereof comprising:
a) obtaining transaminase and bilirubin levels of the patient wherein the transaminase and bilirubin levels are measured within six month prior to the treatment; and
b) administering to the patient a pharmaceutically effective amount of teriflunomide or a pharmaceutically acceptable salt thereof.

6. The method according to claim 5, further comprising:
c) monitoring liver enzymes during the treatment; and
d) if significant liver injury is suspected or if the elevation of liver enzymes greater than three times the upper limit of normal is confirmed, then considering discontinuing the treatment.

7. A method of managing the risk of high blood pressure to allow an effective and safe use of teriflunomide or a pharmaceutically acceptable salt thereof in the treatment of multiple sclerosis in a patient in need thereof comprising:
a) administering to the patient a pharmaceutically effective amount of teriflunomide or a pharmaceutically acceptable salt thereof; and
b) monitoring the blood pressure of the patient during the treatment.

8. The method according to claim 7, further comprising:
c) when hypertension is diagnosed during the treatment, administering to the patient a drug which is known to be effective for treating hypertension.

9. A method for treating multiple sclerosis in a patient having multiple sclerosis and an active acute or chronic infection in need thereof, comprising:
a) resolving the acute or chronic infection prior to the initiation of the treatment; and
b) then administering to the patient a pharmaceutically effective amount of teriflunomide or a pharmaceutically acceptable salt thereof.

10. A method of managing the risk of a serious infection to allow an effective and safe use of teriflunomide or a pharmaceutically acceptable salt thereof in the treatment of multiple sclerosis in a patient in need thereof comprising:
a) administering to the patient a pharmaceutically effective amount of teriflunomide or a pharmaceutically acceptable salt thereof;
b) if the patient develops a serious infection during the treatment, then the treatment should be suspended; and
c) reassessing the benefits and risks prior to re-initiation of the treatment.

11. A method of managing the risk of a bone marrow disorder to allow an effective and safe use of teriflunomide or a pharmaceutically acceptable salt thereof in the treatment of multiple sclerosis in a patient in need thereof comprising:
a) obtaining a complete blood count of the patient wherein the complete blood count is measured with six month prior to the treatment;
b) administering to the patient a pharmaceutically effective amount of teriflunomide or a pharmaceutically acceptable salt thereof;
c) monitoring complete blood count of the patient during the treatment.

12. The method according to claim 11, further comprising:
d) if neutropenia is diagnosed with the patient during the treatment with teriflunomide, the treatment should be considered to be suspended; and
e) if the treatment is suspended, optionally reinitiating the treatment after the complete blood count of the patient is back to normal level.

13. A method of managing the risk of polyneuropathy to allow an effective and safe use of teriflunomide or a pharmaceutically acceptable salt thereof in the treatment of multiple sclerosis in a patient in need thereof comprising:
a) administering to the patient a pharmaceutically effective amount of teriflunomide or a pharmaceutically acceptable salt thereof; and
b) if polyneuropathy is diagnosed during the treatment, then the treatment should be discontinued.

14. An article of manufacture comprising:
a) a packaging material;
b) teriflunomide or a pharmaceutically acceptable salt thereof; and
c) a label or package insert contained within the packaging material indicating at least one messages selected from:
i) liver enzymes should be monitored during the treatment with teriflunomide;
ii) if significant liver injury is confirmed or if the elevation of liver transaminases exceeds three times the upper limit of normal, then the treatment with teriflunomide should be discontinue;
iii) blood pressure should be monitored during treatment with teriflunomide;
iv) women of childbearing potential must use effective contraception to avoid pregnancy while taking teriflunomide;
v) patients with an active acute or chronic infection should not start treatment with teriflunomide until the infection is resolved;
vi) if the patient develops a serious infection during the treatment, then the treatment should be suspended and the benefits and risks should be assessed prior to re-initiation of the treatment;
vii) complete blood count should be periodically monitored during treatment with teriflunomide;
viii) the diagnosis of polyneuropathy is confirmed, the treatment with teriflunomide should be discontinued; and
iv) teriflunomide should not be given to a patient who is hypersensitive to teriflunomide.

15. A package comprising teriflunomide or a pharmaceutically acceptable salt thereof, and a label, wherein the label comprises one or more messages selected from:
a) liver enzymes should be monitored during the treatment with teriflunomide;
b) if significant liver injury is confirmed or if the elevation of liver transaminases exceeds three times the upper limit of normal, then the treatment with teriflunomide should be discontinue;
c) blood pressure should be monitored during treatment with teriflunomide;
d) women of childbearing potential must use effective contraception to avoid pregnancy while taking teriflunomide;
e) patients with an active acute or chronic infection should not start treatment with teriflunomide until the infection is resolved;
f) if the patient develops a serious infection during the treatment, then the treatment should be suspended and the benefits and risks should be assessed prior to re-initiation of the treatment;
g) complete blood count should be periodically monitored during treatment with teriflunomide;
h) the diagnosis of polyneuropathy is confirmed, the treatment with teriflunomide should be discontinued; and
i) teriflunomide should not be given to a patient who is hypersensitive to teriflunomide.

16. A method of promoting the use of teriflunomide or a pharmaceutically acceptable salt thereof, comprising conveying to a recipient at least one message selected from:
a) liver enzymes should be monitored during the treatment with teriflunomide;
b) if significant liver injury is confirmed or if the elevation of liver transaminases exceeds three times the upper limit of normal, then the treatment with teriflunomide should be discontinue;
c) blood pressure should be monitored during treatment with teriflunomide;
d) women of childbearing potential must use effective contraception to avoid pregnancy while taking teriflunomide;
e) patients with an active acute or chronic infection should not start treatment with teriflunomide until the infection is resolved;
f) if the patient develops a serious infection during the treatment, then the treatment should be suspended and the benefits and risks should be assessed prior to re-initiation of the treatment;
g) complete blood count should be periodically monitored during treatment with teriflunomide;
h) the diagnosis of polyneuropathy is confirmed, the treatment with teriflunomide should be discontinued; and
i) teriflunomide should not be given to a patient who is hypersensitive to teriflunomide.

17. A method of providing teriflunomide or a pharmaceutically acceptable salt thereof, wherein teriflunomide or the pharmaceutically acceptable salt thereof is provided along with information indicating that:
a) liver enzymes should be monitored during the treatment with teriflunomide;
b) if significant liver injury is confirmed or if the elevation of liver transaminases exceeds three times the upper limit of normal, then the treatment with teriflunomide should be discontinue;
c) blood pressure should be monitored during treatment with teriflunomide;
d) women of childbearing potential must use effective contraception to avoid pregnancy while taking teriflunomide;
e) patients with an active acute or chronic infection should not start treatment with teriflunomide until the infection is resolved;
f) if the patient develops a serious infection during the treatment, then the treatment should be suspended and the benefits and risks should be assessed prior to re-initiation of the treatment;
g) complete blood count should be periodically monitored during treatment with teriflunomide;
h) the diagnosis of polyneuropathy is confirmed, the treatment with teriflunomide should be discontinued; and
i) teriflunomide should not be given to a patient who is hypersensitive to teriflunomide.
